(19) United States
(12) Patent Application Publication
(10) Pub. No.: US 2015/0377815 A1
(43) Pub. Date: Dec. 31, 2015

(54) NANOTUBE SENSORS FOR CONDUCTING SOLUTIONS

(71) Applicant: EMPIRE TECHNOLOGY DEVELOPMENT LLC, Wilmington, DE (US)

(72) Inventors: Christopher A. WIKLOF, Everett, WA (US); Mordehai MARGALIT, Zichron Yaaqov (IL); Takahisa KUSUURA, Kanagawa (JP); Bradley Kirk ROBERTS, Seattle, WA (US)

(21) Appl. No.: 14/769,370

(22) PCT Filed: Feb. 20, 2013
(86) PCT No.: PCT/US13/26867

§ 371 Date: Aug. 20, 2015

(51) Int. Cl. G01N 27/22; G01N 33/487

(52) U.S. Cl. CPC G01N 27/221; G01N 33/48707

(57) ABSTRACT

Sensors for detecting at least one electrolyte in a conductive solution are described. The sensors may include a dielectric substrate and a resonator having a resonance characteristic and configured to generate a signal in response to an interrogation signal. The resonator may include a conductive layer in contact with the dielectric substrate, at least one layer of nanotubes provided on the conductive layer, and a dielectric layer at least partially encapsulating the nanotubes.

### BACKGROUND

Clinical analysis can involve detection of specific electrolytes (small molecules, antigens, antibodies, proteins, and so forth) in a solution (e.g. blood sample). Electrolytes may be characterized by their charge and mobility in a particular solvent at a particular pH. This information may be helpful in detection, yet detection with specificity and selectivity sufficient for clinical samples remains a challenge, particular in a solution having multiple electrolytes.

### SUMMARY

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

In one embodiment, a sensor configured to detect at least one electrolyte in a conductive solution is described. The sensor may include a dielectric substrate and a first resonator including a conductive layer in contact with the dielectric substrate, at least one layer of nanotubes provided on the conductive layer, and a first dielectric layer provided on the at least one layer of nanotubes such that at least a portion of the nanotubes is not covered by the first dielectric layer, and a second dielectric layer provided on the first dielectric layer such that the second dielectric layer cover a portion of the nanotubes not covered by the first dielectric layer. The first resonator may be configured to generate a response signal to an interrogation signal. The response signal may be indicative of a resonance characteristic of the first resonator which identifies at least one electrolyte.

In one embodiment, a system for detecting at least one electrolyte in a conductive solution is described. The system may include a signal generator, at least one sensor, and at least one detector. The signal generator may be configured to provide an interrogation signal. The at least one sensor is configured to detect at least one electrolyte in the conductive solution and may include a dielectric substrate, and a first resonator that includes a conductive layer in contact with the dielectric substrate, at least one layer of nanotubes provided on the conductive layer, a first dielectric layer provided on the at least one layer of nanotubes such that at least a portion of the nanotubes is not covered by the first dielectric layer, and a second dielectric layer provided on the first dielectric layer such that the second dielectric layer covers a portion of the nanotubes not covered by the first dielectric layer. The first resonator may be configured to generate a response signal to an interrogation signal. The response signal may be indicative of a resonance characteristic of the first resonator which identifies at least one electrolyte. The at least one detector is configured to receive the response signal and generate a decision signal that indicates the resonance characteristic of the first resonator identifying the at least one electrolyte.

In one embodiment, a method for identifying at least one electrolyte in a conductive solution is described. The method may include applying one or more interrogation signals to a first resonator that includes nanotubes, measuring at least one resonant response of the first resonator when excited by the one or more interrogation signals, and determining an identity of at least electrolyte as a function of the at least one resonant response.

### BRIEF DESCRIPTION OF DRAWINGS

In the present disclosure, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

FIG. 1 depicts an illustrative schematic of a sensor for detecting at least one electrolyte in a conductive solution according to an embodiment.

FIG. 1A depicts an illustrative schematic of a sensor for detecting at least one electrolyte in a conductive solution having more than one resonator according to an embodiment.

FIG. 1B depicts an illustrative schematic of an alternate sensor for detecting at least one electrolyte in a conductive solution having more than one resonator according to an embodiment.

FIG. 2 depicts an illustrative schematic of a system for detecting at least one electrolyte in a conductive solution according to an embodiment.

FIG. 3 depicts an illustrative schematic of a process of making a sensor for detecting at least one electrolyte in a conductive solution according to an embodiment.

FIG. 3A depicts an illustrative flow chart for a method of making a sensor for detecting at least one electrolyte in a conductive solution according to an embodiment.

FIG. 4 depicts an illustrative flow chart for a method for identifying at least one electrolyte in a conductive solution according to an embodiment.

FIG. 4A depicts an illustrative flow chart for an alternate method for identifying at least one electrolyte in a conductive solution according to an embodiment.

### DETAILED DESCRIPTION

Described herein are devices, systems, and methods generally related to detecting a presence and/or concentration of at least one electrolyte in a conductive solution. As such, some devices may include a sensor including at least one resonator having resonance characteristic that identifies at least one electrolyte, provided on a dielectric substrate. A typical resonator may include a conductive layer in contact with the dielectric substrate, at least one layer of nanotubes provided on the conductive layer, and at least a dielectric layer at least partially encapsulating the nanotubes. The resonator is configured to generate an electromagnetic response signal in response to an electromagnetic interrogation signal.

The resonator may have a base resonant frequency that is, among other things, determined by the conductive layer and may depend on factors such as, for example, electrical conductivity and geometry of the conductive layer. The layer of nanotubes may modulate the base frequency depending on electrical properties of the nanotubes, size of the nanotube, variation in size of the nanotubes, and/or the like. The base frequency may be further modulated to produce a response signal in response to interaction between the nanotubes and the one or more electrolytes present in a conductive solution in which the resonator is placed. The response signal may differ from the interrogation signal in one or more of a variety of characteristics such as, for example, resonance frequency, phase change, amplitude, Q-factor, band-width, shift in resonance frequency, and the like. This difference may be a result of the interaction of the nanotubes with a particular electrolyte in the conductive solution, and may depend on one or more properties of the electrolyte including, but not limited to, charge, size, mass, concentration, mobility in given solvent, radius of hydration (when water is a solvent), and the like. Each electrolyte interacting with the nanotubes will, typically, produce a particular resonance frequency shift in the resonator. The magnitude of the shift may be indicative of the particular electrolyte, and the amplitude of the response signal at the particular shifted frequency may be indicative of the concentration of the particular electrolyte causing the frequency shift. As such, a single resonator may be configured to detect a variety of electrolytes present in a conductive solution to which the sensor may be exposed.

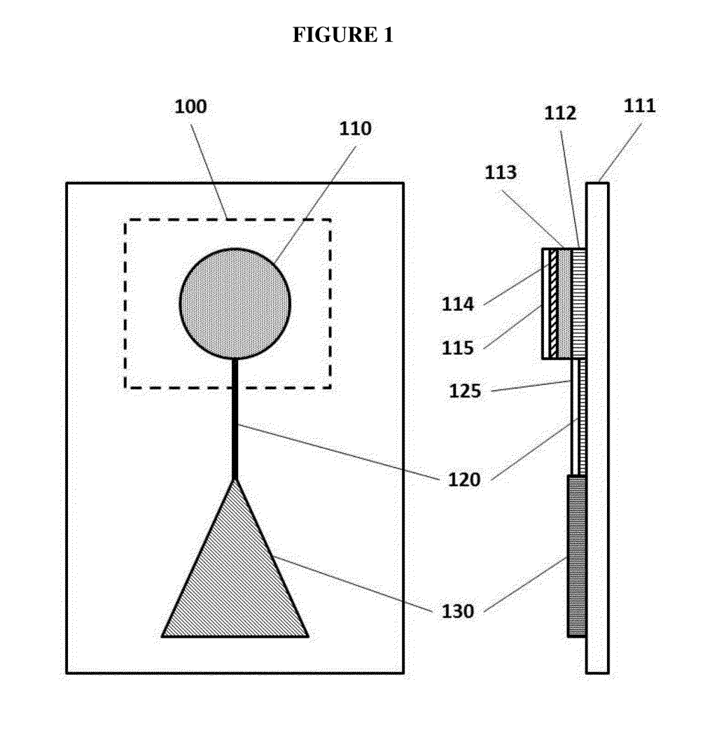

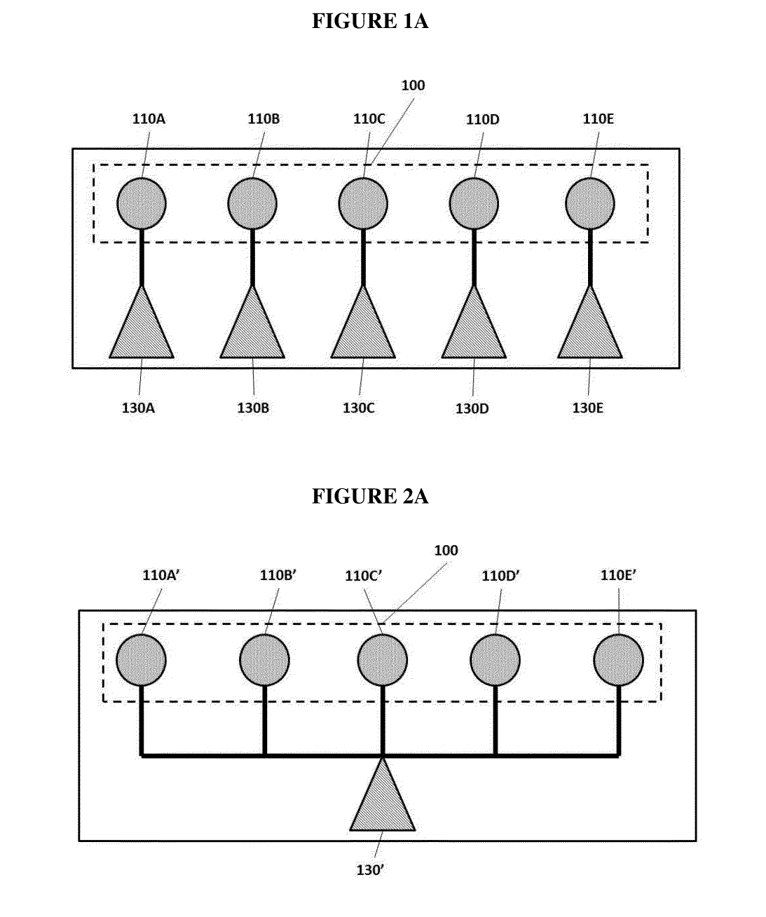

FIG. 1 depicts an illustrative schematic of a sensor for detecting at least one electrolyte in a conductive solution according to an embodiment. The sensor 100 includes a dielectric substrate 111, and a resonator 110. The resonator 110 may include a conductive layer 112, at least one layer 113 of nanotubes provided on the conductive layer 112, and a first dielectric layer 114 provided on the at least one layer 113 of nanotubes such that at least a portion of the nanotubes is not covered by the first dielectric layer 114. In some embodiments, the resonator 110 may further include a second dielectric layer 115 provided on the first dielectric layer 114 such that the second dielectric layer 115 covers a portion of the nanotubes not covered by the first dielectric layer 114. The resonator 110 may be configured to generate a response signal to an interrogation signal. The response signal may be indicative of a resonance characteristic of the first resonator which identifies at least one electrolyte.

Individual components of the sensor 100 may composed of any suitable materials known in the art. For example, the dielectric substrate 111 may be composed of materials including, silicon dioxide, silicon nitride, quartz, glass, polyethylene, polypropylene, polystyrene, polycarbonate, polymethyl methacralate (PMMA), rubber, epoxy, silicone, polydimethyl siloxane (PDMS), and the like, or combinations thereof. In certain embodiments, the conductive layer 112 may be a metal such as, for example, copper, aluminum, chromium, gold, silver, platinum, palladium, and the like, alloys thereof, or combinations thereof. Typically, a base resonance frequency of the resonator 110 is determined by, among other things, geometry and composition of the conductive layer. As such, the geometry of the conductive layer may be varied depending on the desired resonance characteristic of the resonator. In various embodiments, the conductive layer 112 may be provided on the dielectric substrate 111 using any method known in the art.

The layer of nanotubes 113, may have any nanotubes known in the art such as, for example, doped or undoped nanotubes, single-walled nanotubes, multi-walled nanotubes, carbon nanotubes, tungsten disulfide nanotubes, vanadium oxide nanotubes, manganese oxide nanotubes, zinc oxide nanotubes, tin sulfide nanotubes, titanium dioxide nanotubes, DNA nanotubes, and the like, or combinations thereof. The choice of particular nanotube may be based on factors such as, for example, particular electrolytes to be detected, stability, compatibility with fabricating techniques, economy, ability to obtain a uniform geometric distribution where necessary, and the like.

The layer of nanotubes may be aligned in any configuration known in the art. In some embodiments, the nanotubes may be aligned such that length of individual nanotubes extends perpendicular to a plane of the conductive layer 112. Nanotubes in such configuration are typically referred to as vertically aligned nanotubes. In certain embodiments, the nanotubes may be aligned such that length of individual nanotubes extends along the plane of the conductive layer 112. In particular embodiments, the nanotubes may be distributed randomly, and in certain embodiments, the nanotubes may be aligned such that lengths of individual nanotubes are along the same direction. In some embodiments, multiple layers of nanotubes may be provided on the conductive layer 112.

Typically, nanotubes may modulate a resonance characteristic of the resonator 110 depending on their dimension. In general, individual nanotubes attached to the conductive layer 112 can be thought of as antennas, each having a resonance frequency. As such, variability in dimensions of individual nanotubes determines a distribution of resonance frequencies of the resonator 110. In various embodiments, it may be desirable to have a narrow spread of the frequency distribution of resonance of the resonator for distinguishing an interrogation signal from a response signal. For example, in embodiments where presence of an electrolyte shifts the resonance frequency of the resonator 110 by a relatively small amount, a narrow frequency distribution allows for easier detection of the frequency shift. As such, in some embodiments, it may be desirable to have the nanotubes of substantially the same geometry, substantially the same length, or substantially the same diameter.

Without wishing to be bound by theory, a high permittivity of a conductive solution causes charge transfer from the nanotubes to the conductive solution, resulting in loss of resonance. Thus, it may be desirable in some embodiments, to provide a dielectric layer 114 on the layer 113 of nanotubes to avoid loss of resonance from transfer of charge to the conductive solution. However, such a dielectric layer 114 may limit an interaction between the nanotubes and electrolytes of the conductive solution. As such, at least a portion of the nanotubes may not be covered by the dielectric layer 114 to allow an interaction between the layer 113 of nanotubes and electrolytes of the conductive solution. In some embodiments, the portion of the nanotubes not covered by the dielectric layer 114 may be about 1%, about 5%, about 10%, about 15%, about 20%, about 25%, about 30%, about 35%, about 40%, about 45%, about 50%, or any value between any two of these values, of the length of the nanotubes. In various embodiments, the dielectric layer 114 may be of any material known in the art such as, for example, silicone, PDMS, PMMA, polystyrene, poly(methyl acralate) (PMA), polyimide, polynorbornenes, benzocyclobutene, polytetrafluoroethylene (PTFE, or Teflon), hydrogen silsesquioxane (HSQ), methylsilsesquioxane (MSQ), SU-8 epoxy, or the like, or combinations thereof.

In general, a suitably provided first dielectric layer 114 may provide for adequate interaction between electrolytes of the conductive solution and the layer of nanotubes 113, while reducing a loss of resonance caused by transfer of charge to the conductive solution. However, in some embodiments, it may be desirable to provide a second dielectric layer 115 to further reduce the transfer of charge to improve quality of the response signal. In various embodiments, a second dielectric layer 115 may be provided on the first dielectric layer 114 such that the second dielectric layer 115 covers the portion of the layer of nanotube 113 not covered by the first dielectric layer 114. The second dielectric may be chosen to further reduce transfer of charge while allowing an interaction between the electrolytes of the conductive solution and the nanotubes. Any dielectric having a low permittivity may be used as a second dielectric. In various embodiments, the second dielectric may be, for example, a silane, a silicone, silicon dioxide, titanium dioxide, HSQ, MSQ, and the like, or combinations thereof.

When an interrogation signal is applied to the resonator 110 with no electrolytes present, the resonator 110 may generate a base response signal. The base response signal is characterized by one or more characteristics including, but not limited to, one or more resonant frequencies, a phase, a Q-factor, a band-width, an amplification factor, and the like. When the resonator 110 is exposed to a conductive solution having electrolytes, in various embodiments, interaction between the layer of nanotubes 113 and electrolytes may modulate one or more characteristics of the response signal generated in response to an interrogation signal. In some embodiments, the interaction between electrolytes and the nanotubes may cause a shift in resonance frequency of the resonator 110, which may depend on factors such as, for example, charge, size, mass, concentration, mobility in given solvent, radius of hydration (when water is a solvent), and the like. As such, the magnitude of frequency shift may be indicative of presence of a particular electrolyte and in such embodiments, the amplitude of the response signal at the shifted frequency may be indicative of a concentration of the electrolyte. In certain embodiments, a Q-factor at a shifted frequency may be indicative of a concentration of the electrolyte. In some embodiments, the response signal in presence of electrolytes may have substantially the same frequency as the base response signal, however, the electrolytes may cause a phase-shift, such that magnitude of phase-shift may be indicative of an electrolyte and an amplitude of the response signal at a shifted phase may be indicative of concentration of the electrolyte. As such, various permutations of resonance characteristics may be indicative of the electrolyte and its concentration in various embodiments of the sensor 100.

In some embodiments, a signal generator 130 may be used for providing the interrogation signal. The signal generator 130 could be any signal generator known in the art such as, for example, an analog signal generator, a digital signal generator, an oscilloscope, and the like, or combinations thereof. The interrogation signal may be any signal known in the art such as, for example, a sine wave signal, a sawtooth signal, a step signal, a triangular signal, an arbitrary waveform signal, and the like, or combinations thereof, and may have a frequency of about 100 Hz to about 100 GHz. The interrogation signal may have a frequency of about 100 Hz, about 1 KHz, about 10 KHz, about 100 KHz, about 1 MHz, about 10 MHz, about 100 MHz, about 1 GHz, about 10 GHz, about 100 GHz, or any frequency or range of frequencies between any two of these values.

In various embodiments, the sensor may include more than one resonator. FIGS. 1A, and 1B depict sensors for detecting at least one electrolyte in a conductive solution having more than one resonators according to some embodiments. In some embodiments, the more than one resonators 110A′-E′ may all receive an interrogation from one signal generator 130′. In such embodiments, each of the more than one resonators 110A % E′ is configured such that modulation of its response characteristics by a particular electrolyte is more prominent than modulation of its response characteristics by any other electrolyte. Thus, in such embodiments, each of the more than one resonators 110A′-E′ may be configured to detect a single electrolyte. In certain embodiments, the signal generator 130′ may provide multiple interrogation signals separately corresponding to the base response signals for the more than one resonators 110A-E. In some embodiments, the signal generator 130′ may provide a single interrogation signal that is a combination of the base response signals for the more than one resonators 110A-E. In various embodiments, the interrogation signal may include one or more harmonics and/or sub-harmonics of a particular fundamental frequency, such that the particular fundamental frequency corresponds to a base response signal of a given resonator.

In some embodiments, more than one signal generators 130A-E may provide the interrogation signal to the more than one resonators 110A-E such that each of the more than one resonator receives a different interrogation signal depending on its response characteristics from one of the corresponding signal generators 130A-E. In various embodiments, the interrogation signals received by each of the more than one resonator 110A-E may be different, each resonator identifying one particular electrolyte.

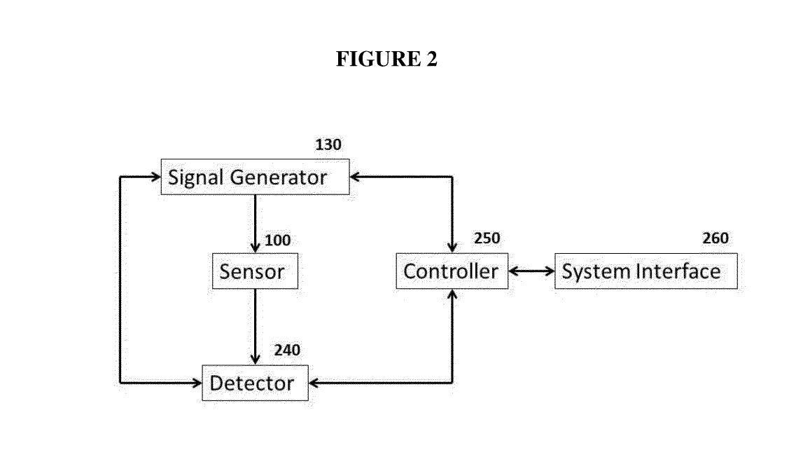

Embodiments are directed to a system for detecting at least one electrolyte in a conductive solution. FIG. 2 depicts an illustrative schematic of a system for detecting at least one electrolyte in a conductive solution according to an embodiment. In various embodiments, the system may include a signal generator 130, at least one sensor 100, and at least one detector 240. The signal generator 130 may be configured to provide an interrogation signal. The at least one sensor 100 is configured to detect at least one electrolyte in the conductive solution and may include a dielectric substrate, and a first resonator that includes a conductive layer in contact with the dielectric substrate, at least one layer of nanotubes provided on the conductive layer, a first dielectric layer provided on the at least one layer of nanotubes such that at least a portion of the nanotubes is not covered by the first dielectric layer, and a second dielectric layer provided on the first dielectric layer such that the second dielectric layer covers a portion of the nanotubes not covered by the first dielectric layer. The first resonator may be configured to generate a response signal to an interrogation signal. The response signal may be indicative of a resonance characteristic of the first resonator which identifies at least one electrolyte. The at least one detector is configured to receive the response signal and generate a decision signal that indicates the resonance characteristic of the first resonator identifying the at least one electrolyte.

In some embodiments, the system may further include a controller 250 that is operably connected to the at least one detector 240 and configured to receive the detection signal and compare the detection signal with an expected value to determine the presence or absence of the at least one electrolyte. In some embodiments, the signal generator, the at least one detector and the controller are part of a system interface 260.

In various embodiments, the at least one sensor may be coupled with the signal generator and/or the at least one detector by any means known in the art including, but not limited to, wireless coupling and wired coupling. In some embodiments, for example, the sensor may be inductively coupled with the signal generator and/or the at least one detector. In certain embodiments, the sensor may be connected to a coil which is inductively coupled to a second coil connected to the signal generator and/or the at least one detector. In some embodiments, the sensor may be coupled with the signal generator and/or the at least one detector using, for example, a coaxial cable, a microstrip, a stripline, a balanced line, a twisted pair, a twin-lead, a lecher line, and the like, or combinations thereof.

In some embodiments, the system may further include at least one control sensor having a control resonator. The control sensor may be associated with a conductive solution having a known electrolyte. The control resonator is configured to generate a control response signal in response to the interrogation signal. The control response signal is indicative of a resonance characteristic of the control resonator when the at least one control sensor senses the known electrolyte such that the resonance characteristic of the control resonator identifies the known electrolyte. The known electrolyte could be any electrolyte known in the art. Illustrative examples include, but are not limited to, H⁺, K⁺, Na⁺, Cl⁻, Ag⁺, Cu⁺, Cu²⁺, Hg²⁺, and the like. In certain embodiments, the control sensor may be used for calibrating the at least one sensor used in the system. In some such embodiments, the system may further include at least one controller configured to compare the resonance characteristic of the control resonator to the resonance characteristic of the first resonator to identify a difference indicative of the presence of the at least one electrolyte about the at least one sensor. The identified difference may correspond to one or more of a difference in any resonance characteristic known in the art or described herein such as, for example, amplitude, Q-factor, phase, resonant frequency, shift in resonance frequency, and the like.

In various embodiments, the system may include one or more sensors described herein. In particular embodiments, the sensor may have carbon nanotubes, and the resonator may be configured to have a resonance frequency that shifts in presence of the at least one electrolyte present in the conductive solution when the second dielectric layer comes in contact with the conductive solution.

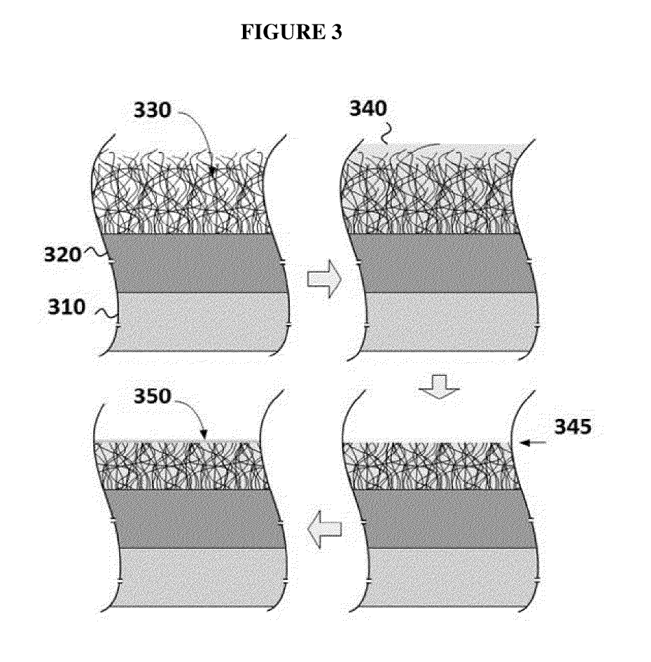

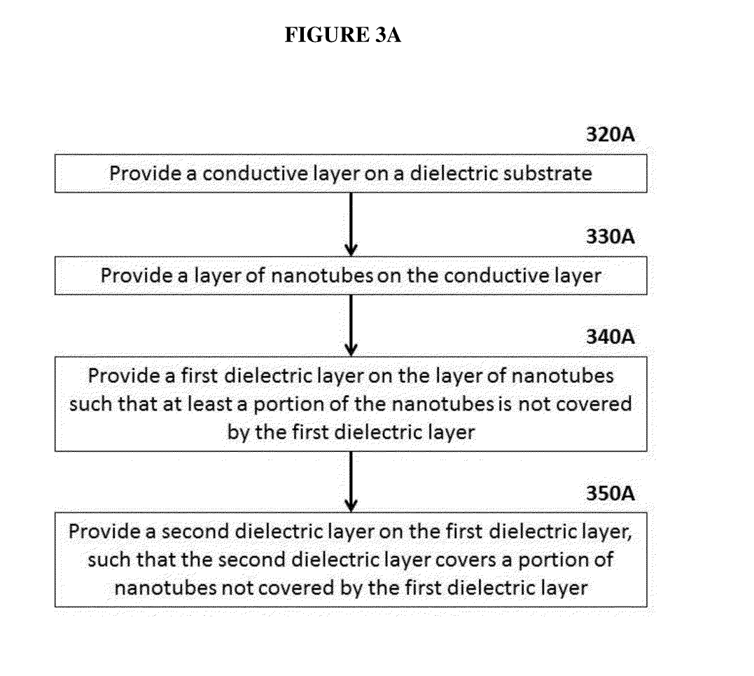

Further embodiments are directed to methods of making a sensor configured to detect at least one electrolyte in a conductive solution. FIG. 3 depicts an illustrative schematic of a process of making a sensor for detecting at least one electrolyte in a conductive solution according to an embodiment, and FIG. 3A depicts an illustrative flow chart for a method of making a sensor for detecting at least one electrolyte in a conductive solution according to an embodiment. In some embodiments, a method for making a sensor configured to detect at least one electrolyte in a conductive solution may include providing 320A a conductive layer 320 on a dielectric substrate 310, providing 330A a layer of nanotubes 330 on the conductive layer 320, providing 340A a first dielectric layer 340 on the layer of nanotubes such that at least a portion of the nanotubes is not covered by the first dielectric layer 340, and providing 350A a second dielectric layer 350 on the first dielectric layer 340 such that the second dielectric layer 350 covers a portion of nanotubes 330 not covered by the first dielectric layer 340.

In various embodiments, providing 320A the conductive layer 320 on the dielectric substrate 310 may include attaching the conductive layer 320 to the dielectric substrate 310 using any method known in the art such as, for example, using a bonding agent, using an adhesive layer, using a solder agent, and the like, or combinations thereof. In some embodiments, providing 320A the conductive layer 320 may include depositing a conductive layer on the dielectric substrate 310 using, for example, electroplating, sputtering, thermal evaporation, electron-beam evaporation, pulsed laser deposition, or a combination thereof. The dielectric substrate 310 could be any suitable dielectric known in the art as described herein. Similarly, the conductive layer 320 used for making the sensor can be any conductor known in the art as described herein.

Providing 330A a layer of nanotubes 330 may, in certain embodiments, include vapor based deposition techniques such as, for example, various types of chemical vapor deposition, thermal evaporation, vapor phase epitaxy, and the like. In some embodiments, providing 330A a layer of nanotubes 330 may include coating, spin-coating, dipping, spraying, printing, and the like, or combinations thereof, using a suitable solution and/or suspension of nanotubes. In various embodiments, any nanotubes known in the art may be used for making the sensor as described herein and will determine the particular processes used for providing the nanotubes.

Providing 340A a first dielectric layer 340 may include, in various embodiments, steps such as, for example, spraying, spin-coating, dip-coating, vapor deposition, self-assembly, and the like, or combinations thereof. Similarly, providing 350A a second dielectric layer 350, in various embodiments, include, without limitation, steps such as spraying, spin-coating, dip-coating, vapor deposition, self-assembly, and the like, or combinations thereof. In certain embodiments, providing the first dielectric layer and providing the second dielectric layer may further include addition of a curing agent and/or a cross-linking agent, heat-curing, photo-curing, annealing, and the like, or combinations thereof. In various embodiments, the first and the second dielectric layers may be of any suitable dielectric known in the art and as described herein, and will determine the particular processes used for providing the layers. In certain embodiments, it may be desirable, depending on the particular process being used, to provide the first dielectric layer 340 such as to encapsulate the layer of nanotubes 330. In such embodiments, the method for making the sensor may include a step for removing a portion of the first dielectric layer (as depicted by 345) to uncover at least a portion of the nanotubes. As such, any suitable process known in the art may be used for removing a portion of the first dielectric layer. Illustrative examples of such process include, without limitation, etching, cutting using a microtome, ablation, plasma assisted oxidation, and the like, or a combination thereof. A skilled artisan will appreciate that, in general, certain processes are more desirable over others depending on the particular materials in use.

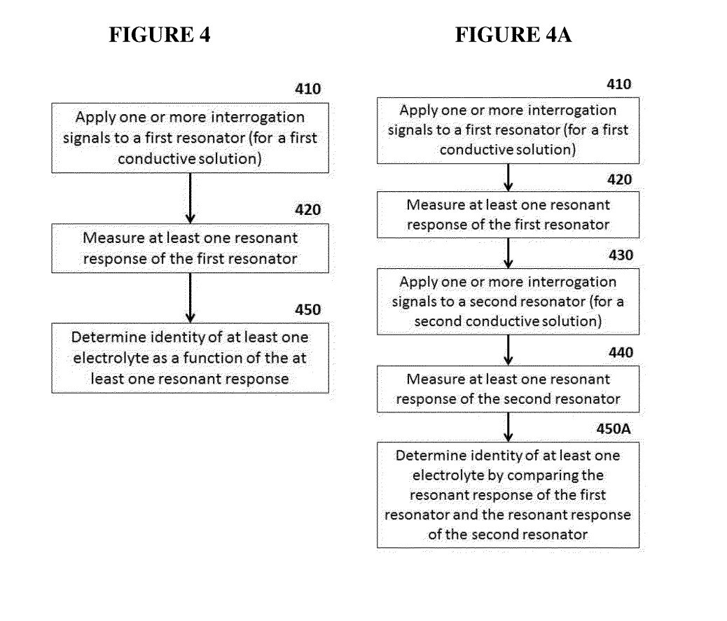

Embodiments are further directed to methods for identifying at least one electrolyte in a conductive solution. FIGS. 4 and 4A depict illustrative flow charts for example methods for identifying at least one electrolyte in a conductive solution. In various embodiments, a method for identifying at least one electrolyte in a conductive solution may include applying 410 one or more interrogation signals to a first resonator that includes nanotubes, measuring 420 at least one resonant response of the first resonator when excited by the one or more interrogation signals, and determining 450 an identity of at least one electrolyte as a function of the at least one resonant response. In some embodiments, the method may further include applying 430 one or more interrogation signals to a second resonator that is associated with a second conductive solution different from the first conductive solution, measuring 440 at least one resonant response of the second resonator when excited by the interrogation signals, and determining 450A an identity of at least one electrolyte by comparing the at least one resonant response of the first resonator and the at least one resonant response of the second resonator. In various embodiments, the second resonator may be used to calibrate the first resonator.

In certain embodiments, the first resonator and/or the second resonator may include any nanotubes known in the art. Various embodiments of resonators are described herein. In some embodiments, the resonators may include a conductive layer provided on a dielectric substrate, at least one layer of nanotubes provided on the conductive layer, a first dielectric layer provided on the layer of nanotubes such that at least a portion of the nanotubes is not covered by the first dielectric layer, and a second dielectric layer provided on the first dielectric layer such that the second dielectric covers a portion of the nanotubes not covered by the first dielectric layer. The resonator is configured such that a resonance characteristic of the resonator identifies at least one electrolyte.

The one or more interrogation signals may be any signals known in the art. Examples of various interrogation signals that may be used are described herein. In some embodiments, an interrogation signal may be a sine wave signal, a sawtooth signal, a step signal, a triangular signal, an arbitrary waveform signal, and the like, or combinations thereof, and may have a frequency of about 100 Hz to about 100 GHz. The interrogation signal may have a frequency of about 100 Hz, about 1 KHz, about 10 KHz, about 100 KHz, about 1 MHz, about 10 MHz, about 100 MHz, about 1 GHz, about 10 GHz, about 100 GHz, or any frequency or range of frequencies between any two of these values.

A resonant response of the first resonator and/or the second resonator, in some embodiments, may include, for example, a shift in resonant frequency, a change in the Q-factor, a phase-shift, a change in amplitude, a change in band-width, a change in amplification factor, and the like, or a combination thereof.

### EXAMPLES

### Example 1

### Electrolyte Sensor with Carbon Nanotubes

A copper circle having a thickness of about 400 μm and a diameter of about 200 μm is electrodeposited on an epoxy substrate. A layer of vertically aligned carbon nanotubes with a length of about 200 nm is grown on top of the copper surface using chemical vapor deposition. A layer of PDMS having a thickness of about 150 nm is spin-coated on the copper surface such that the layer of carbon nanotubes is partially embedded in the PDMS layer. After curing the PDMS layer, a 55 nm thick layer of HSQ is spin-coated on top of the PDMS layer so as to cover a portion of uncovered carbon nanotubes.

The epoxy substrate is attached on to a metal plate which acts as the ground plane.

### Example 2

### Electrolyte Detection System

A copper circle having a thickness of about 100 μm and a diameter of about 175 μm is sputtered on an epoxy surface. A layer of vertically aligned single-walled carbon nanotubes with an average length of about 150 nm is grown on top of the copper surface using chemical vapor deposition. A 160 nm thick layer of PDMS is spin-coated on the copper surface such that the carbon nanotubes are fully embedded in PDMS. PDMS is then degassed and cured at about 80° C. for about 1-2 hours. The substrate is then sectioned using a microtome to obtain a PDMS thickness of about 140 p.m. This results in carbon nanotubes terminating at the surface of the PDMS layer. A 20 nm thick layer of HSQ is then spin-coated on top of the PDMS layer to form the sensor.

The epoxy substrate is attached on to a metal plate which acts as the ground plane for the sensor.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A sensor configured to detect at least one electrolyte in a conductive solution, the sensor comprising:
a dielectric substrate; and
a first resonator having a resonance characteristic and configured to generate a response signal in response to an interrogation signal, wherein the resonance characteristic of the first resonator identifies at least one electrolyte in the conductive solution, wherein the first resonator includes:
a conductive layer in contact with the dielectric substrate and operably connected to a first signal generator by a first connection, wherein the first signal generator is configured to provide the interrogation signal,
at least one layer of nanotubes provided on the conductive layer,
a first dielectric layer provided on the at least one layer of nanotubes such that at least a portion of the nanotubes is not covered by the first dielectric layer, and
a second dielectric layer provided on the first dielectric layer such that the second dielectric layer covers a portion of the nanotubes not covered by the first dielectric layer.

2. (canceled)

3. The sensor of claim 1, wherein the at least one layer of nanotubes comprises at least one of a monolayer of one or more doped or undoped nanotubes, single-walled nanotubes, multi-walled nanotubes, carbon nanotubes, tungsten disulfide nanotubes, vanadium oxide nanotubes, manganese oxide nanotubes, zinc oxide nanotubes, tin sulfide nanotubes, titanium dioxide nanotubes, DNA nanotubes, and vertically aligned nanotubes.

4. (canceled)

5. The sensor of claim 1, wherein the nanotubes have substantially the same diameter.

6. The sensor of claim 1, wherein the nanotubes have substantially the same length.

7. The sensor of claim 1, wherein the nanotubes are aligned perpendicular to a plane of the conductive layer.

8. The sensor of claim 1, wherein the conductive layer comprises at least one of copper, aluminum, gold, silver, chromium, palladium, and platinum.

9. The sensor of claim 1, wherein:
the first dielectric layer comprises at least one of silicone, PDMS, PMMA, polystyrene, poly(methyl acralate) (PMA), polyimide, polynorbornenes, benzocyclobutene, polytetrafluoroethylene (PTFE, or Teflon), hydrogen silsesquioxane (HSQ), methylsilsesquioxane (MSQ), and SU-8 epoxy, and
the second dielectric layer is a monolayer comprises at least one of a silane, a silicone, silicon dioxide, titanium dioxide, HSQ and MSQ.

10. (canceled)

11. The sensor of claim 1, wherein the resonance characteristic of the first resonator comprises one or more of a resonant frequency of the first resonator, a frequency shift in the resonant frequency of the first resonator, a Q-factor associated with the first resonator, an amplitude associated with the response signal, a phase associated with the response signal, or a difference in a plurality of resonant frequencies.

12. (canceled)

13. The sensor of claim 1, further comprising at least one second resonator, wherein a resonance characteristic of the at least one second resonator is different from the resonance characteristic of the first resonator.

14. The sensor of claim 13, wherein the at least one second resonator is operably connected to at least one second signal generator.

15. The sensor of claim 13, wherein the at least one second resonator is operably connected to the first signal generator via a second connection.

16. The sensor of claim 13, wherein an interrogation signal associated with the at least one second resonator is different from the interrogation signal associated with the first resonator.

17. A system for detecting at least one electrolyte in a conductive solution, the system comprising:
a signal generator configured to provide an interrogation signal;
at least one sensor configured to detect at least one electrolyte in the conductive solution, the at least one sensor comprising:
a dielectric substrate, and
a first resonator having a resonance characteristic and configured to generate a response signal in response to an interrogation signal,
wherein the first resonator comprises a conductive layer in contact with the dielectric substrate, at least one layer of nanotubes provided on the conductive layer, a first dielectric layer provided on the at least one layer of nanotubes such that at least a portion of the nanotubes is not covered by the first dielectric layer, and a second dielectric layer provided on the first dielectric layer such that the second dielectric layer covers a portion of the nanotubes not covered by the first dielectric layer,
wherein the resonance characteristic of the first resonator identifies the at least one electrolyte; and
at least one detector configured to receive the response signal and generate a detection signal that indicates the resonance characteristic of the first resonator identifying the at least one electrolyte.

18. The system of claim 17, wherein the signal generator and the at least one detector are part of a system interface.

19. The system of claim 17, further comprising a controller that is operably connected to the at least one detector and configured to receive the detection signal and compare the detection signal with an expected value to determine the presence or absence of the at least one electrolyte.

20. The system of claim 17, wherein the at least one sensor is wirelessly coupled to one of the signal generator and the at least one detector.

21. The system of claim 17, further comprising:
at least one control sensor including a control resonator,
wherein the at least one control sensor is associated with a conductive solution having a known electrolyte, and
wherein the control resonator is configured to generate a control response signal in response to the interrogation signal, the control response signal being indicative of a resonance characteristic of the control resonator when the at least one control sensor senses the known electrolyte such that the resonance characteristic of the control resonator identifies the known electrolyte; and
at least one controller configured to compare the resonance characteristic of the control resonator to the resonance characteristic of the first resonator to identify a difference indicative of the presence of the at least one electrolyte about the at least one sensor,
wherein the identified difference corresponds to at least one of a difference in amplitude, a difference in Q-factor, a difference in phase, a difference in resonant frequency, a shift in resonance frequency, or a difference in a plurality of resonant frequencies.

22. (canceled)

23. (canceled)

24. The system of claim 17, wherein the first resonator further comprises a layer of carbon nanotubes provided on a conductive layer, a first dielectric layer at least partially encapsulating the carbon nanotubes and a second dielectric layer provided on the first dielectric layer such that the second dielectric layer is in contact with the conductive solution, wherein the resonator has a resonance frequency that shifts in presence of the at least one electrolyte.

25. A method for making a sensor configured to detect at least one electrolyte in a conductive solution, the method comprising:
providing a conductive layer on a dielectric substrate;
providing a layer of nanotubes on the conductive layer;
providing a first dielectric layer on the layer of nanotubes;
removing a portion of the first dielectric layer such that at least a portion of the nanotubes is not covered by the first dielectric layer; and
providing a second dielectric layer on the first dielectric layer, such that the second dielectric layer covers a portion of nanotubes not covered by the first dielectric layer.

26. (canceled)

27. The method of claim 25, wherein removing a portion of the first dielectric layer comprises removing the portion of the first dielectric layer by at least one of etching and cutting using a microtome.

28. The method of claim 25, wherein providing the conductive layer comprises attaching a conductive layer to the dielectric substrate using at least one of a bonding agent, an adhesive layer, and a solder agent.

29. The method of claim 25, wherein providing the conductive layer comprises depositing a conductive layer on the dielectric substrate using at least one of electroplating, sputtering, thermal evaporation, electron-beam evaporation, and pulsed laser deposition.

30. The method of claim 25, wherein providing the layer of nanotubes comprises providing the layer of nanotubes by at least one of vapor based deposition, coating, dipping, spraying, spin-coating, printing, or a combination thereof.

31. The method of claim 25, wherein providing the first dielectric layer comprises providing the first dielectric layer by at least one of spraying, spin-coating, dip-coating, vapor deposition, self-assembly, adding a curing agent, adding a cross-linking agent, heat-curing, photo-curing, and annealing.

32. The method of claim 25, wherein providing the second dielectric layer comprises providing the second dielectric layer by at least one of spraying, spin-coating, dip-coating, vapor deposition, self-assembly, adding a curing agent, adding a cross-linking agent, heat-curing, photo-curing, and annealing.

33. (canceled)

34. A method for identifying at least one electrolyte in a first conductive solution, the method comprising:
applying one or more interrogation signals to a first resonator, wherein the first resonator includes nanotubes;
measuring at least one resonant response of the first resonator when excited by the one or more interrogation signals;
applying one or more interrogation signals to a second resonator that is associated with a second conductive solution different from the first conductive solution;
measuring at least one resonant response of the second resonator when excited by the interrogation signals; and
determining an identity of at least one electrolyte by comparing the at least one resonant response of the first resonator and the at least one resonant response of the second resonator.

35. (canceled)
